(19) United States
(12) Patent Application Publication
(10) Pub. No.: US 2024/0149019 A1
(43) Pub. Date: May 9, 2024

(54) MULTIPURPOSE EXTENSION CATHETER

(71) Applicant: Nurlan Toktaganovich ISMAILOV, Kyzylorda (KZ)

(72) Inventors: Nurlan Toktaganovich ISMAILOV, Kyzylorda (KZ); Bolatbek Bimendeyevich BAIMAKHANOV, Almaty (KZ); Sabit Bekseitovich PAZILOV, Almaty (KZ); Nuridin Yegemberdievich AMITOV, Kyzylorda (KZ); Turganbai MAKHANOV, Kzyzlorda (KZ); Nariman Boltayevich TABYNBAYEV, Almaty (KZ); Berik Ruslanovich DAUTBAYEV, Almaty (KZ); Azamat Kosherbayevich KOZHAKHMETOV, Kyzylorda (KZ); Nurbol Sabitovich BAZARBAYEV, Kyzylorda (KZ); Nurlykhan Torekhanuly KASSYMOV, Kyzylorda (KZ); Kenesary Aituganovich BASPAYEV, Ayteke bi (KZ); Kanat Akmaganbetovich NABDINOV, Kyzylorda (KZ)

(21) Appl. No.: 18/281,957

(22) PCT Filed: Mar. 2, 2022
(86) PCT No.: PCT/KZ2022/000001

§ 371 Date: Sep. 14, 2023

(30) Foreign Application Priority Data
Jul. 24, 2021 (KZ) 2021/0731.2

(51) Int. Cl. A61M 25/00; A61M 25/09

(52) U.S. Cl. CPC A61M 25/0069; A61M 25/09; A61M 2025/0046; A61M 2025/0681

(57) ABSTRACT

The invention relates to medical technology, and more particularly to multipurpose extension catheters. A multipurpose extension catheter configured in the form of an elongate flexible body with a through-channel thereinside has a rectilinear proximal part and a straight or bent distal part for entering the aorta, the distal end of the guide catheter terminating in a soft tip. The curvature of the distal part of the catheter is in keeping with standard types of guide catheters, which correspond to the dimensions of the aortic arch and the type of origin of the coronary arteries and peripheral vessels. The flexible body of the catheter has a lumen for a guidewire controlled by a torquer, and the tip of the catheter is provided with a sleeve, wherein, between the sleeve and the tip of the catheter, a hydrophilic lubricant is provided inside the sleeve to allow unhindered movement of the sleeve for passage of the catheter through a stenosis (defect), improved control of the guidewire inside the flexible body by the torquer and improved and easier movement of inserted devices through the catheter to the site of a vessel defect.

The invention relates to medical technology, namely to multi-purpose catheter extenders, and can be used for easy and safe delivery of various devices (e.g., a stent or balloon) inside the catheter to the area of the vascular lesion (stenosis), including through complex areas of the coronary and peripheral vessels (arteries and veins) with various bends.

The closest analogue in technical essence to the claimed invention is a coronary guiding catheter, which is made in the form of an elongated flexible body with a through channel inside and having a proximal straight part and a distal curved part for entering the aorta with a final soft tip at the distal end of the said guiding catheter, wherein the curvature of the distal portion of the catheter corresponds to standard types of guiding catheters matching the size of the aortic arch and the type of branching of the coronary arteries and peripheral vessels. A lateral opening is made in the distal part of the catheter. The lateral opening is closed with an elastic membrane with a slit/RU 168379 U1, published 31 Jan. 2017.

The disadvantages of this analogue are that the catheter is not able to pass through stenoses (lesions), instead the catheter is simply placed in the ostial part of the vessel and other devices (balloon, stent, etc.) are introduced.

The objective of the invention is to create a new improved design of a multi-purpose catheter-extender with improved technical characteristics.

The technical result is the possibility to pass the catheter through stenoses (lesions), to improve the controllability of the guidewire torquer in the flexible body, to improve and facilitate the advancement of the introduced devices (e.g., a stent or a balloon) through the catheter to the vascular lesion (e.g., stenosis) thanks to the sheath.

This is achieved by providing a multi-purpose catheter extender in the form of an elongated flexible body with an internal channel and having a proximal straight portion and a distal straight or curved portion for entering the aorta with a terminating soft tip at the distal end of said guiding catheter, wherein the curvature of the distal portion of the catheter corresponds to standard types of guiding catheters matching the size of the aortic arch and the type of branching of the coronary arteries and peripheral vessels. According to the invention, the flexible body of the catheter has a lumen for a guidewire guided by a torquer, and the tip of the catheter is provided with a sheath, with hydrophilic lubricant between the sheath and the tip of the catheter inside the sheath for unimpeded movement of the sheath.

The outer diameter of the flexible catheter body is from 1.92 to 2 mm inclusive, and the inner diameter of the flexible catheter body is from 1.83 to 1.9 inclusive. The sheath is made of silicone, latex or polytetrafluoroethylene and is 15 to 34 mm long.

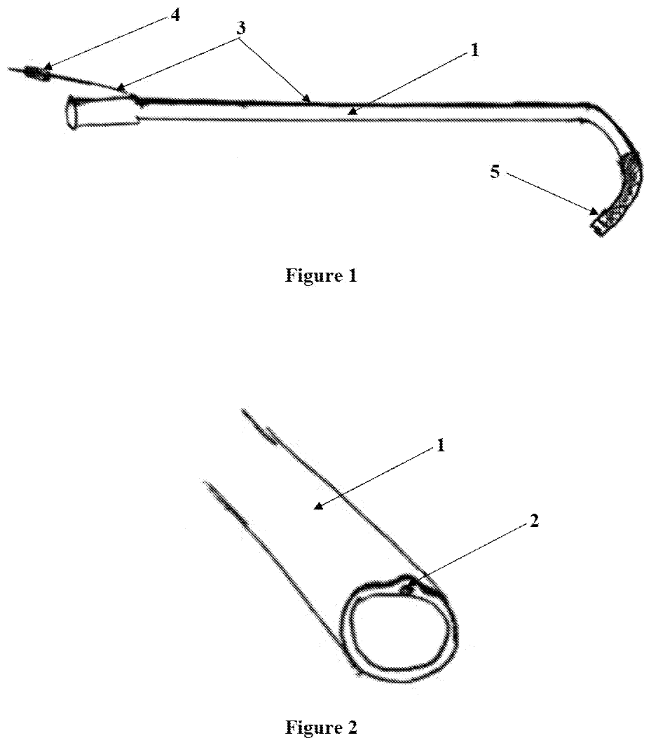

FIG. 1 shows a schematic diagram of a multi-purpose catheter extender.

FIG. 2 shows a schematic diagram of the flexible catheter body showing the lumen for the guidewire.

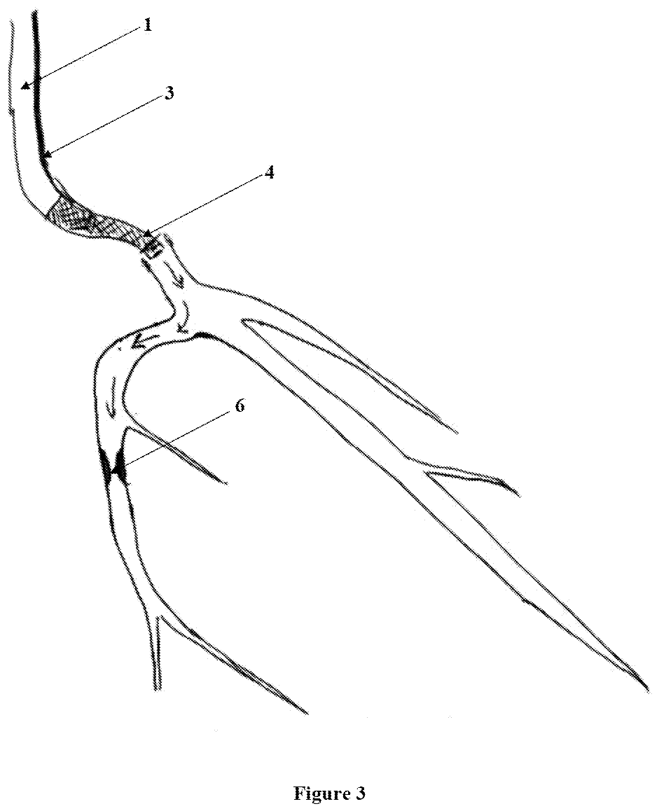

FIG. 3 shows a schematic diagram of a coronary vessel with the catheter inserted.

FIGS. 1-3 show the following elements:

1—flexible catheter body; 2—lumen for the guidewire; 3—catheter guidewire; 4—torquer for guiding the guidewire; 5—sheath at the tip of the flexible body; 6—lesion site (e.g., stenosis).

A multi-purpose catheter-extender is made in the form of an elongated flexible body (1) with an internal channel having a lumen (2) for a guidewire (3) controlled by a torquer (4), and the tip of the catheter is provided with a sheath (5), and between the sheath (5) and the tip of the catheter there is a hydrophilic lubricant for unimpeded movement of the sheath. The outer diameter of the flexible catheter body (1) is from 1.98 to 2 mm inclusive, and the inner diameter of the flexible catheter body (1) is from 1.83 to 1.9 inclusive.

The invention operates as follows.

### EXAMPLE

If there is a lesion (e.g., stenosis) of a vessel (artery, vein) with complicated access (e.g., sharp bends in the vessel), a coronary guiding catheter (outer diameter in the range of 1.98-2 mm and inner diameter in the range of 1.83-1.9 mm) is inserted into the target artery, the body of which has a lumen for a torqued guidewire. The sheath at the tip of the catheter allows the torqued catheter to be easily and quickly guided into difficult areas of the vessel and ensures easy delivery of various devices (stent or balloon) through the catheter to the coronary lesion site.

1. A multi-purpose catheter extender made in the form of an elongated flexible body with an internal channel and having a proximal straight portion and a distal straight or curved portion for entering the aorta with a terminating soft tip at the distal end of said guiding catheter, the curvature of the distal portion of the catheter being in accordance with standard types of guiding catheters, characterised in that the flexible body of the catheter has a lumen for a torquer-guided guidewire and the tip of the catheter is provided with a sheath, with a hydrophilic lubricant between the sheath and the tip of the catheter for unimpeded movement of the sheath.

2. According to claim 1, the catheter extender is characterised in that the outer diameter of the flexible body of the catheter is between 1.98 and 2 mm inclusive.

3. According to claim 1, the catheter extender is characterised in that the inner diameter of the flexible catheter body is from 1.83 to 1.9 inclusive.

4. According to claim 1, the catheter extender is characterised in that the sheath is made of silicone or polytetrafluoroethylene with a length of 15 to 30 mm.
